(19) United States
(12) Patent Application Publication
(10) Pub. No.: US 2026/0331290 A1
(43) Pub. Date: Oct. 22, 2026

(54) AN AI BASED SYSTEM FOR MONITORING OF PERIPHERAL VASCULAR DISEASE PATIENTS

(71) Applicant: BIOTRONIK AG, Buelach (CH)

(72) Inventor: Azadeh Mehrabi, Meilen (CH)

(21) Appl. No.: 19/119,516

(22) PCT Filed: Oct. 19, 2023
(86) PCT No.: PCT/EP2023/079161

§ 371 Date: Apr. 9, 2025

(30) Foreign Application Priority Data
Nov. 15, 2022 (EP) 22207531.9

(51) Int. Cl. G16H 50/30; G06T 7/00

(52) U.S. Cl. CPC G16H 50/30; G06T 7/0016; G06T 2207/10016; G06T 2207/30088; G06T 2207/30096

(57) ABSTRACT

A device for monitoring a health risk for a patient. The device obtains visual information associated with a skin of the patient. The device detects an anomaly on the skin of the patient based at least in part on the obtained visual information. The device assigns a scoring value to the detected anomaly. The scoring value is associated with the health risk for the patient.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/079161, which was filed Oct. 19, 2023, which application claimed priority from EP Application Serial No. 22207531.9, which was filed Nov. 15, 2022.

### FIELD OF THE INVENTION

A field of the invention concerns health condition monitoring devices.

### BACKGROUND

Changing life habits over the recent years have led to an increase in a variety of different dysfunctions of the circulatory system with respective hazards for a health state of a patient. Burdens on the health of the patient can lead to increased treatment costs, increased demand for medical staff, etc.

These dysfunctions of the circulatory system are often caused by damage, occlusion and/or inflammation of arteries and/or veins of the patient. These dysfunctions, inter alia, relate to peripheral vascular diseases (PVD) and represent a major cause of morbidity and mortality. Peripheral arterial disease (PAD), chronic venous disease (CVD), which includes chronic venous insufficiency (CVI) and deep vein thrombosis (DVT), are common types of PVDs and are the most prevalent in the lower extremities.

Early diagnosis and treatment of the such diseases is crucial to address the risk of these dysfunction and avoid mortality and morbidity of diseased patients. Unfortunately, 50% of people suffering from one of the aforementioned diseases are asymptomatic and therefore often fail to seek medical assistance and/or are generally not screened by clinicians in the absence of a diagnosed disease as there can not be an obvious reason for a detailed screening.

Symptoms of PAD are often not recognized by the patients as severe symptoms for PAD or a deterioration of a known existing PAD disease.

Effective and efficient clinical non-invasive diagnostic tools are needed to determine vascular diseases, in particular, for asymptomatic PVD and PAD patients who have the same risk of morbidity and mortality as those with more obvious symptoms. While noninvasive methods have been developed to at least support the diagnosis of PVDs and PADs, the currently existing methods for diagnosing and curing PVDs and PADs can require many manual and thus time-intensive diagnosis steps) that limit their effectiveness. This creates the risk of failure to diagnose especially in view of the rising number of patients with a PVD and/or a PAD and the shortcomings of current methods.

Typical approaches to diagnose PAD, as used in the art, are shown and compared in the table below.

TABLE 1
overview of different approaches to diagnose a PAD and/or a PVD.

| diagnosis method | method | looks at |
|---|---|---|
| Doppler ultrasound | sound waves for imaging | blood flow in vessels |
| ankle-brachial index | ultrasound and blood | comparison of blood |
| (ABI) | pressure cuff around | pressure readings in leg |
| | ankle and arm, measured | and arm, as lower |
| | before and during | pressure in leg could |
| | exercise | indicate a blockage |
| angiography | injected dye in a catheter | flow of dye through |
| | that is guided through the | blood vessels to diagnose |
| | artery | f clogged artery |
| magnetic resonance | magnetic field imaging | image of blood vessels to |
| angiography (MRA) | | diagnose blockage |
| computed | X-ray imaging | image of blood vessels to |
| tomography | | diagnose blockage |
| angiography (CTA) | | |

### SUMMARY OF THE INVENTION

A device according to a preferred embodiment monitors a health risk for a patient. The device obtains visual information associated with a skin of the patient. The device detects an anomaly on the skin of the patient based at least in part on the obtained visual information. The device assigns a scoring value to the detected anomaly. The scoring value is associated with the health risk for the patient.

### BRIEF DESCRIPTION OF THE DRAWINGS

The following figures are provided to support the understanding of the present invention:

FIG. 1: Illustration of an exemplary procedure to monitor a health risk for a patient;

FIG. 2: Illustration of exemplary claudication locations;

FIG. 3: Illustration of exemplary locations for determining a pulse strength of a patient;

FIG. 4: Exemplary system overview according to aspects of the present invention;

FIG. 5: Exemplary flow chart of a method for anomaly detection in visual information;

FIG. 6: Illustration of an exemplary neural network configured to act as an autoencoder;

FIGS. 7A and 7B: Illustration of an exemplary method for anomaly detection in visual information according to aspects of the present invention;

FIG. 8: Exemplary flowchart for assigning a scoring value to visual information obtained from two sides of a body of a patient.

### DETAILED DESCRIPTION OF THE PREFERRED EMBODIMENTS

Preferred embodiment devices overcome drawbacks of prior devices The device can include a module for obtaining visual information associated with a skin of the patient and a module for detecting an anomaly on the skin of the patient based at least in part on the obtained visual information. Moreover, the device can include a module for assigning a scoring value to the detected anomaly, wherein the scoring value is associated with the health risk for the patient.

The device can be implemented as a remote entity such as, e.g., a server, but also as a local entity, such as a smartphone, for example.

The visual information can include information of a locally restricted area of the skin of the patient (e.g., a locally restricted area of the skin at the neck of the patient and/or a locally restricted area of the skin at an upper leg of the patient and/or any other (suitable) locally restricted area of the skin of the patient). The locally restricted area can, e.g., be between 1 to 30 cm², preferably between 5 to 25 cm², more preferably between 10 and 20 cm² or any other suitable area.

An anomaly can be understood as a feature derivable from the visual information indicative for a health risk for the patient. Additionally or alternatively, an anomaly can be understood as a feature in the obtained visual information associated with a diseased patient which is not present in respective visual information associated with a patient considered as healthy.

The scoring value can be provided as a number (e.g., an integer number) within a predefined numerical interval which can indicate a likelihood that the patient suffers from a certain heath risk. As an example, the scoring value can be any number between 0 and 100, wherein the number can indicate a likelihood (in percent) that the patient suffers from a certain health risk. Alternatively, it can also be possible that the scoring value spans the interval of 0 to 10, wherein 0 can be an indication for the likelihood that the patient does (most likely) not suffer from a certain health risk whereas 10 can be understood as an indication for the likelihood that the patient (most likely) suffers from a certain health risk.

In a preferred embodiment, the module for obtaining visual information is a module for continuously obtaining visual information and/or the module for detecting an anomaly is a module for continuously detecting an anomaly and/or the module for assigning a scoring value is a module for continuously assigning a scoring value. Accordingly, a tighter monitoring of the health risk of the patient can be achieved.

This device according to an aspect of the present invention can allow a noninvasive diagnostic and/or monitoring tool for a potential health risk for a patient. The device can be used by a patient (e.g., due to its simplicity and/or its preferably compact form factor) to track and recognize existing symptoms of the patient or as a tracking tool for physicians to gain access to the progressive symptoms of a potentially prevalent disease. This can allow an early diagnosis of a disease and can thus support a better and safe treatment of the patient with respective decreased costs for a health system (e.g., as a potential treatment can be initialized in an early stage of a progressing disease). Since a gathering of visual information, according to an aspect of the present invention, can be done by the patient alone, the burden on doctors (e.g., in terms of workload) and other members of a medical staff which can commonly be part of the diagnosis procedure, can be reduced as a main part of the diagnoses can be substituted by the interplay between the device, for monitoring a health risk for the patient, and the patient without the need for a doctor. Moreover, the risk that a patient can not realize a potential hazard arising from a potentially present health risk can be minimized and the potential health risk can be determined before it can become an emergency.

Another aspect of the present invention relates to a further device for monitoring a health risk for a patient. The device can include a module for obtaining visual information associated with a skin of the patient and optionally a module for detecting an anomaly on the skin of the patient based at least in part on the obtained visual information. Moreover, the device can include a module for receiving a scoring value to be assigned to the detected anomaly, wherein the scoring value is associated with the health risk for the patient.

The module for obtaining visual information can include a sensor configured to acquire visual information associated with the skin of the patient. In some exemplary cases, the sensor can be provided as a camera, e.g. including a CCD chip.

The module for receiving the scoring value can include a module for communicating with a remote entity such that the scoring value can be received from the remote entity. The module for communication can also include module for transmitting the visual information to another device, e.g. a server. The another device can then detect the anomaly and/or assign the scoring value to the anomaly. In some exemplary cases, the device can include module for determining the scoring value at least partially locally, e.g., by the device itself. The another device, respectively, the module for determining the scoring value can be configured to execute an algorithm which determines or assists in determining the scoring value.

By providing the device as outlined above, a portable device can be provided for monitoring a health risk for a patient. Notably, by providing the device with a module for receiving a scoring value, the device can not necessarily be provided with a module for performing the calculation of the scoring value and/or detecting the anomaly which can be accompanied by a respective high energy demand, which can disadvantageously affect a battery lifetime of the device. Therefore, by outsourcing the calculation of a scoring value, the battery lifetime of the device can be extended, and the device can be provided with a more compact formfactor. Additionally, the manufacturing costs of the device can be decreased.

The device for monitoring a health risk for a patient can be adapted to monitor a prevalence, presence and/or progression of a peripheral arterial disease, PAD, and/or a peripheral vascular disease, PVD, and/or a diabetes.

The device for monitoring a health risk for a patient can be adapted to monitor a likelihood, a degree of progression, a probability of having, not having, developing and/or not developing PAD, PVD and/or diabetes and/or a likelihood of complications from PAD, PVD and/or diabetes.

The monitoring of a prevalence can include a monitoring of a likelihood whether a patient suffers from one or more of the aforementioned diseases and/or whether an existing disease has deteriorated over time.

By configuring the device for monitoring at least one or more of the aforementioned health risks for the patient, an early diagnosis and/or treatment of a potential prevalence of at least one of said diseases can be supported. This can in particular be advantageous since the prevalence of said diseases can oftentimes progress without any symptoms or with only a negligible deterioration of potentially existing symptoms such that the prevalence and/or a deterioration of any of said diseases can not be recognized during an early stage. The device can thus advantageously contribute to a decrease of the morbidity of diseased patients.

The visual information can be a video sequence.

A video sequence can relate to a video sequence with a duration of 1-10 s, preferably of 2-8 s, more preferably of 5-6 s. In some cases, the video sequence can be a color video. In alternative cases, the video can be a black and white video. Additionally or alternatively, the visual information can include a series of photographs.

The video sequence can in some cases additionally include one or more freeze images.

By providing the visual information as a video sequence, a larger skin area (as compared to a single freeze image) of the skin can be provided. This can allow a monitoring of respective larger area of the skin of the patient and can thus more likely support an early diagnosis (and preferably a respective early treatment of a prevailing disease) as a potential small anomaly can less likely be overlooked.

The module for detecting the anomaly can be adapted to detect a lesion at at least one location on the skin of the patient and preferably an appearance of the lesion.

A lesion can be understood as any damage or abnormal change of the skin of the patient. This can include a wound, a hematoma, an ulcer, a discoloration, a pigmentation, combinations therefrom or any other lesion which can be associated with a health risk for a patient.

An appearance can be understood as a dimension of the lesion (e.g., a maximum (e.g., a top to bottom) extension in length units and/or a surface area of the lesion), a color and/or a color change of the lesion (as compared to a previously detected anomaly) and/or any other visually acquirable parameters which can be associated with a health risk for the patient.

This can advantageously contribute to a non-invasive monitoring of a health risk for a patient as only visual information, derivable from a skin of the patient, can be required for the monitoring. This can simplify the monitoring as such, can decrease the overall monitoring costs (e.g., as no complex medical equipment can be required and/or since no physically present doctors can be required), can allow a gapless monitoring (e.g., since a monitoring of a health state of the patient can not be limited to distinct screenings by, e.g., a doctor) of a health state of a patient and can thus advantageously contribute to an improved health monitoring of the patient.

The module for obtaining can include a module for receiving the visual information from a local device.

The device for monitoring a health risk for a patient can be physically separated from the patient and/or the local device, i.e., the device for monitoring can not be in close vicinity to the patient and/or the local device and can instead be located in another room as the patient and/or another building and/or can be accessible by a network and/or internet connection.

The module for receiving can include a module for communicating with the local device by a wired connection and/or preferably by wireless connection (e.g., Bluetooth, WiFi, LTE, 5G, etc.).

The module for obtaining can further include a module for decompressing a received visual information in case the visual information is transmitted in a compressed manner.

By implementing the device for monitoring such that it includes a module for receiving the visual information, the device for monitoring can support the implementation of the device as a remote entity such as, e.g., a server. This can facilitate an at least partially centralized data warehouse system at which received visual information can be collected and/or analyzed. Therefore, a centralized data analysis can be facilitated. This can additionally allow that the main computing power for detecting an anomaly and/or for assigning a scoring value can be provided by the device for monitoring such that the local device can not be required to be provided with dedicated data storage capabilities and/or can not be adapted with a dedicated computation power for detecting the anomaly and/or for assigning the scoring value. This can allow that the local device can be adapted with a compact form factor, decreased manufacturing costs and an extended battery lifetime.

In a preferred embodiment, the module for obtaining visual information are configured to obtain a first visual information associated with a first part of the skin and a second visual information associated with a second part of the skin, the module for detecting an anomaly are configured to detect a first anomaly on the first part of the skin at least in part on the obtained first visual information, and a second anomaly on the second part of the skin at least in part on the obtained second visual information, and the module for assigning a scoring value are configured to assign a first scoring value to the first anomaly and a second scoring value to the second anomaly. The device further includes a module for making a first comparison between the first scoring value and the second scoring value, and a module for assigning a first comparison scoring value to the first anomaly based on the first comparison.

The first part of the skin is distinct from the second part of the skin. For instance, the first part of the skin can be part of the left leg of the patient, whereas the second part of the skin is part of the right leg.

In case the first part of the skin includes an anomaly, but the second part of the skin is healthy and does not include an anomaly (the second anomaly is rather within a range of anomalies associated with healthy skin), the device provides a more accurate and robust determination of a health risk to a patient by comparing healthy skin with skin potentially including an anomaly.

By the comparison between the first scoring value which is a scoring value and thus associated to the health risk of the patient, and the second scoring value, which is a scoring value and thus associated with the health risk of the patient, the first comparison scoring value is associated with the health risk of the patient and allows a more accurate and robust determination of a health risk to a patient.

In a further preferred embodiment, the module for obtaining visual information are further configured to obtain a third visual information associated with the first part of the skin and a fourth visual information associated with the second part of the skin, wherein the third visual information and the fourth visual information are obtained later in time than the first visual information, respectively, the second visual information. The module for detecting an anomaly are further configured to detect a third anomaly on the first part of the skin at least in part on the obtained third visual information, and a fourth anomaly on the second part of the skin at least in part on the obtained fourth visual information, the module for assigning a scoring value are configured to assign a third scoring value to the third anomaly and a fourth scoring value to the fourth anomaly, the module for making the first comparison are further configured to make a second comparison between the first scoring value and the third scoring value, and a third comparison between the second scoring value and the fourth scoring value, and/or a fourth comparison between the third scoring value and the fourth scoring value and the module for assigning the first comparison scoring value are further configured to assign a second comparison scoring value to the third anomaly based on the second comparison and the third comparison and/or the first comparison and/or the fourth comparison.

The third and the fourth visual information are obtained at some time later than the first and second visual information in order to capture a potential progression of the potential anomaly. A reasonable time between obtaining the first, respectively, second visual information and the third, respectively, the fourth visual information can be hours, days, weeks or even months. In an even further preferred embodiment, the module for obtaining visual information are continuously obtaining visual information of the first and/or second part of the skin at more than two points in time. According to this embodiment, the progression of an anomaly in time can be determined and compared to the progression of healthy skin over time. Thus, the device provides an even more accurate and robust determination of a health risk to a patient.

In an alternative embodiment, the module for obtaining visual information are configured to obtain a first visual information and a second visual information, wherein the first visual information and the second visual information are associated with a part of the skin of the patient and the second visual information is obtained later in time than the first visual information. The module for detecting an anomaly are configured to detect a first anomaly on the part of the skin at least in part on the obtained first visual information, and a second anomaly on the part of the skin at least in part on the obtained second visual information, and the module for assigning a scoring value are configured to assign a first scoring value to the first anomaly and a second scoring value to the second anomaly. The device further includes module for making a comparison between the first scoring value and the second scoring value, and module for assigning a comparison scoring value to the second anomaly based on the comparison.

The second visual information is obtained at some time later than the first visual information in order to capture a potential progression of the potential anomaly. A preferrable duration of the time period between obtaining the first visual information and the second visual information can be hours, days, weeks or even months. In an even further preferred embodiment, the module for obtaining visual information are configured to continuously obtain visual information of the part of the skin. According to this embodiment, the temporal evolution or the progression of an anomaly can be determined.

By the comparison between the first scoring value which is a scoring value and thus associated to the health risk of the patient, and the second scoring value, which is a scoring value and thus associated with the health risk of the patient, the comparison scoring value is associated with the health risk of the patient and allows a more accurate and robust determination of the health risk to the patient as it takes the temporal evolution of the anomaly into account.

The first and second comparison scoring value can also be provided as a number (e.g., an integer number) within a predefined numerical interval which can indicate a likelihood that the patient suffers from a certain health risk. As an example, also the first and second comparison scoring value can be any number between 0 and 100, wherein the number can indicate a likelihood (in percent) that the patient suffers from a certain health risk. Alternatively, it can also be possible that the first and second comparison scoring value spans the interval of 0 to 10, wherein 0 can be an indication for the likelihood that the patient does (most likely) not suffer from a certain health risk whereas 10 can be understood as an indication for the likelihood that the patient (most likely) suffers from a certain health risk.

In a further preferred embodiment, the module for detecting the anomaly on the skin of the patient include an artificial intelligence, AI, engine or model. The AI engine can include an autoencoder (AE), a variational autoencoder (VAE), a generative adversarial network (GAN) or a convolutional neural network (CNN) for an accurate and robust determination of a health risk to a patient.

The device for monitoring a health risk for a patient can further include module for providing the patient with at least one question associated with the health state of the patient, wherein the at least one question is preferably associated with an extent of a pain experienced by the patient and/or a location of the pain experienced by the patient. The device can additionally or alternatively include a module for receiving at least one answer from the patient in response to the at least one question (wherein the question can optionally be provided to the patient by a different device).

An extent of pain can be understood as a ranking of the currently experienced pain by a number. As an example, the currently experienced pain can be indicated by an integer number between 0 and 10, wherein 0 can indicate that currently no pain is experienced wherein 10 can indicate that a very strong pain is experienced. It is emphasized that any other suitable interval (e.g., from 0 to 5) can equally be implemented to rank the pain experienced by the patient.

A location of the pain can refer to an area of the body of the patient at which pain is experienced, e.g., an area including an upper leg of the patient, a neck of the patient, etc.

In cases, in which the device is implemented as a local device, the module for providing the patient with at least one question can include a module for displaying the question (such as, e.g., a graphical display and preferably a graphical user interface (GUI)) and/or can include a module for acoustically providing the patient with the at least one question (such as, e.g., at least one loudspeaker which can provide the question to the patient as a spoken sequence).

In case the device is implemented as a local device, the reply can be received by displaying possible replies to the patient (e.g., on a touch-sensitive display). As an example, after providing the at least one question to the patient (e.g., “Please rank the current extent of pain you are experiencing in your right upper leg”), a scale of 0 to 10 can be provided to the patient wherein the patient can select one of the numbers as a measure for the extent of pain the patient is currently experiencing.

Additionally or alternatively, the module for receiving can further include a microphone. The microphone can be configured to receive a spoken reply to the at least one question from the patient. As an example, in view of the above-mentioned question, the patient can answer “5” as an indication of the extent of pain the patient is currently experiencing in his/her right upper leg.

It can be possible that the received reply is further processed/evaluated at the local device. However, additionally or alternatively, it can also be possible that the received answer is transmitted to a remote entity.

In other cases, preferably if the device is implemented as a remote entity, the module for providing can include a database wherein the database can be provided with the at least one question to be provided to the patient.

In this case, the module for providing can further include a module for communicating with a local device, wherein the device can preferably include a module for transmitting the question to a local device. The local device can then provide the at least one question to the patient as outlined above.

In this case, the module for receiving can include a module for receiving a reply to the at least one question from the local device. The local device can be implemented as outlined above such that it is capable of receiving a respective response to the at least one question.

By providing the patient with at least one question, the monitoring of a health risk for the patient can further be improved as the monitoring (and a potential diagnosis) does not only rely on a single parameter (e.g., visual information) but can further be refined by the at least one question. Therefore, an early and more reliable diagnosis of potentially prevailing health risk for the patient can advantageously be supported.

The device for monitoring a health risk for a patient can further include a module for weighting the received at least one answer and/or the scoring value, respectively, the first scoring value and/or the second scoring value and/or the third scoring value and/or the fourth scoring value and/or the first comparison scoring value and/or the second comparison scoring value to provide an estimation of the prevalence of PAD, PVD and/or diabetes.

The module for weighting can include a module for analyzing the received reply and extracting information from the reply. As an example, the at least one question can include the question “Is the estimated total distance you were walking today more than 200 m or less?”. In response, the patient, can reply “more than 200 m” (“more than 200 m” can be considered as information). Based on the extracted content of the reply, the module for weighting can assign a weighting value (e.g., an integer number between 1 to 4) to the reply, indicating to which extent the received answer and/or the scoring value, respectively, the first scoring value and/or the second scoring value and/or the third scoring value and/or the fourth scoring value and/or the first comparison scoring value and/or the second scoring value can be associated with the prevalence of PAD, PVD and/or diabetes.

More specifically, if the patient replies that pain occurred at rest, a weighting value of 4 can be assigned to the reply, if the patient replies that pain occurred after walking less than 200 m, a weighting value of 3 can be assigned, if the patient replies that pain occurred after walking more than 200 mm, a weighting value of 2 can be assigned and if the patient replies that pain occurred after walking 1 km, a weighting value of 1 can be assigned.

The module for weighting can be implemented at the local device (i.e., the local device can at least partially locally assign the weighting factor to the provided response) and/or can be implemented at a remote device (i.e., the remote device can at least partially assign the weighting factor to the provided response).

By providing the device with a module for weighting, at least one question concerning the health state of the patient can be taken into account (besides the visual information) when monitoring the health state of the patient. The weighting of the at least one question can facilitate providing a probability value (encoded in a total sum of the assigned weighting values associated with the at least one question) indicating a likelihood with which a patient can suffer from PVD, PAD and/or diabetes.

The device for monitoring a health risk for a patient can further include a module for requesting, preferably periodically, visual information of the skin of the patient.

The module for requesting can include a module for outputting a push notification to the patient indicating that new visual information of the skin of the patient shall be supplied. The module for outputting can include a display (if the device is implemented as a local device) such that the push notification can be supplied visually as a message on the display and/or can include an LED, wherein the LED can be configured to flash if new visual information is to be supplied. Additionally or alternatively, the module for outputting can include a loudspeaker such that the indication that new visual information is required can be output as a beeping sound and/or a vocal prompt (e.g., a spoken sequence).

In some cases, the (local) device can internally be configured (e.g., by a hardcoded configuration) to request the visual information.

Additionally or alternatively, the (local) device can be provided with a module to receive a request message from a remote device and can include a module for outputting the received message.

A periodic request can relate to a request which is output once a day, once a week, once a month, once a year and/or at any other suitable interval. The module for requesting can additionally or alternatively be configured to output a request for visual information upon an externally supplied request such as a request provided remotely by a doctor of the patient.

By providing the device with a module for (e.g., periodically) requesting visual information, it can be ensured that a gapless tracking and monitoring of the health state of a patient occurs. This can decrease the risk that a deterioration of the health state of the patient can remain unrecognized.

Another aspect of the present invention relates to a method for monitoring a health risk for a patient. The method can include obtaining visual information associated with a skin of the patient and detecting an anomaly on the skin of the patient based at least in part on the obtained visual information. Moreover, the method can include assigning a scoring value to the detected anomaly, wherein the scoring value is associated with the health risk for the patient.

The visual information, the assigning of a scoring value, the health risk and/or the anomaly can be implemented as outlined above.

Another aspect of the present invention relates to a method for monitoring a health risk for a patient. The method can include obtaining visual information associated with a skin of the patient and, optionally, detecting an anomaly on the skin of the patient based at least in part on the obtained visual information, wherein detecting an anomaly can include comparing the obtained visual information with previous obtained visual information. Moreover, the method can include receiving a scoring value to be assigned to the detected anomaly, wherein the scoring value is associated with the health risk for the patient.

The visual information, the assigning of a scoring value, the health risk and/or the anomaly can be implemented as outlined above.

The method for monitoring a health risk for a patient can further include receiving the visual information at a remote entity, and preferably performing the detecting and/or the assigning at least partially at the remote entity.

In such an implementation, the visual information can initially be obtained by a local device (e.g., a camera, a medical diagnostics tool and/or a portable device (e.g., a smartphone), etc.). Subsequent to the obtaining, the obtained information can be provided to a remote device.

By receiving the visual information at the remote entity and preferably performing the detecting and/or the at least partially assigning at the remote entity, computation and/or storage intensive tasks can be outsourced from the local device to the remote entity. This can allow to extend the battery lifetime of the local device and can generally allow a more compact form factor for the portable device. Moreover, by outsourcing computation intensive tasks to the remote entity (which can preferably be optimized for computation intensive tasks), a faster detecting and/or assigning can be supported. Moreover, if the detecting/assigning is performed at the remote entity, the detecting/assigning can be based at least in part on a previously acquired (historic) data set which can further improve the reliability of the detecting and/or the assigning.

In a preferred embodiment, the method for monitoring a health risk for a patient can further include obtaining a second visual information associated with a second part of the skin of the patient, detecting a second anomaly on the skin of the patient based at least in part on the obtained second visual information, assigning a second scoring value to the detected second anomaly, wherein the second scoring value is associated with the health risk for the patient, making a first comparison between the first scoring value and the second scoring value, assigning a first comparison scoring value to the first anomaly based on the first comparison.

In case the first part of the skin includes an anomaly, but the second part of the skin is healthy and does not include an anomaly (the second anomaly is rather within a range of anomalies associated with healthy skin), the method provides a more accurate and robust determination of a health risk to a patient by comparing healthy skin with skin potentially including an anomaly. In case the first part of the skin and the second part of the skin include an anomaly, the method provides an improved determination of the extent of the health risk to a patient. Accordingly, the first comparison scoring value is associated with the health risk for the patient.

Further, the method for monitoring a health risk for a patient can include obtaining a third visual information associated with the first part of the skin of the patient after obtaining the first visual information, detecting a third anomaly on the skin of the patient based at least in part on the obtained third visual information, assigning a third scoring value to the detected third anomaly, wherein the third scoring value is associated with the health risk for the patient, making a second comparison between the first scoring value and the third scoring value, assigning a second comparison scoring value to the third anomaly based on the second comparison.

By comparing the first scoring value and the third scoring value, and assigning a second comparison scoring value, the progression and the temporal evolution of the disease is determined. Thus, the second comparison scoring value can be associated to the health risk for the patient.

In a preferred embodiment, a first period of time of hours, days, weeks or months can lie in between obtaining the third visual information associated with the first part of the skin of the patient and obtaining the first visual information. Thus, the frequency of obtaining visual information can be adjusted to the disease (PAD, PVD and/or diabetes), to the extent and/or progression of the disease.

In another embodiment, the method can include obtaining visual information and/or detecting an anomaly and/or assigning a scoring value in a continuous manner. Accordingly, a tighter monitoring of the health risk of the patient can be achieved.

In a further preferred embodiment, the method can include obtaining a fourth visual information associated with the second part of the skin of the patient after obtaining the second visual information; detecting a fourth anomaly on the skin of the patient based at least in part on the obtained fourth visual information; assigning a fourth scoring value to the detected fourth anomaly, wherein the fourth scoring value is associated with the health risk for the patient; making a third comparison between the second scoring value and the fourth scoring value, and/or a fourth comparison between the third scoring value and the fourth scoring value, and assigning a third comparison scoring value to the third anomaly based on the second comparison and the third comparison and/or the first comparison and/or the fourth comparison.

Accordingly, the embodiment combines the advantages of the embodiment including a comparison between scoring values associated to different parts of the skin and the embodiment including a comparison between scoring values associated with the same part of the skin, but with different points in time.

In a preferred embodiment, a second period of time of hours, days, weeks or months can lie in between obtaining the second visual information associated with the second part of the skin of the patient and obtaining the fourth visual information. In a further preferred embodiment, the first period of time and the second period of time are the same.

The method for monitoring a health risk for a patient can further include providing the patient with at least one question associated with the health state of the patient, wherein the at least one question is preferably associated with an extent of a pain experienced by the patient and a location of the pain experienced by the patient and receiving at least one answer from the patient in response to the at least one question. The method can further include preferably weighting the received at least one answer to provide an estimation of a prevalence of PAD.

In some cases, the patient can only be provided with at least one question when the method is performed for the first time. Additionally or alternatively, the patient can be provided with the at least one question periodically (e.g., once a day, once a week, once a month, once a year, etc.) and/or on an irregular basis (e.g., if a deterioration of an anomaly in the visual information is determined).

The detection of an anomaly can include providing the obtained first visual information, second visual information, third visual information and/or fourth visual information to a trained artificial intelligence, AI, engine, wherein the trained AI engine, preferably including an autoencoder, has been trained on previously obtained visual data considered as healthy and preferably based on a skin type of the patient. The detection can further include deriving a first anomaly in the obtained first visual information by the trained AI engine, preferably by reconstructing the obtained first visual information by the autoencoder and comparing the reconstructed information to the obtained first visual information, and/or deriving a second anomaly in the obtained second visual information by the trained AI engine, preferably by reconstructing the obtained second visual information by the autoencoder and comparing the reconstructed information to the obtained second visual information, and/or deriving a third anomaly in the obtained third visual information by the trained AI engine, preferably by reconstructing the obtained third visual information by the autoencoder and comparing the reconstructed information to the obtained third visual information, and/or deriving a fourth anomaly in the obtained fourth visual information by the trained AI engine, preferably by reconstructing the obtained fourth visual information by the autoencoder and comparing the reconstructed information to the obtained fourth visual information.

The training on previously obtained visual data can be understood as training the AI engine such that it can be capable of reproducing one or more data sets included in the obtained visual data used for training. The AI engine can include a neural network, including an input layer, an output layer and one or more hidden layers in between the input layer and the output layer. Each node of a certain layer can be interconnected to each node of a preceding and/or a subsequent layer. Each interconnection can be provided with a weighting factor. The weighting factors can be determined by training the neural network.

The detection of the anomaly can be based at least in part on determining a difference between the reconstructed obtained visual information and the obtained visual information which potentially includes an anomaly.

The training of the AI engine to a skin type of the patient can allow an adaption of the anomaly detection to different appearances of an anomaly on the skin of the patient relative to the skin of the patient. As an example, in some cases, an anomaly can look different on a colored skin as compared to a Caucasian skin. A training of the AI engine to different skins of the patient can thus support a more precise and reliable determination of an anomaly. It can be a separate aspect of the present invention.

By an AI based determination of an anomaly in the obtained visual information, the reliability of the monitoring can further be improved. This can be the case since the AI engine can be trained to a large data set of images and can thus potentially also be trained to rare and/or small anomalies which can, e.g., be overlooked by a doctor.

Another aspect of the present invention relates to a system for monitoring a health risk for a patient. The system can include one or more of the devices as outlined above.

In a preferred embodiment, the system can include the device configured to act as a remote entity. Furthermore, the system can preferably include the device configured to act as a local entity.

The system can allow an advantageous synergetic interplay of the local device (as described above) and the remote entity (as described above). Such a synergetic interplay can be facilitated by combining one or more advantageous properties of the local device (e.g., portability, easy handling, easy operability, optimized user experienced, low-cost, etc.) with one or more advantageous properties of the remote entity (e.g., computation power, large storage capability, centralized data storage, centralized maintenance, etc.). This can advantageously support a reliable monitoring of a health risk for the patient.

Another aspect of the present invention relates to a computer program including code, which when executed on a computer, performs the method as outlined above.

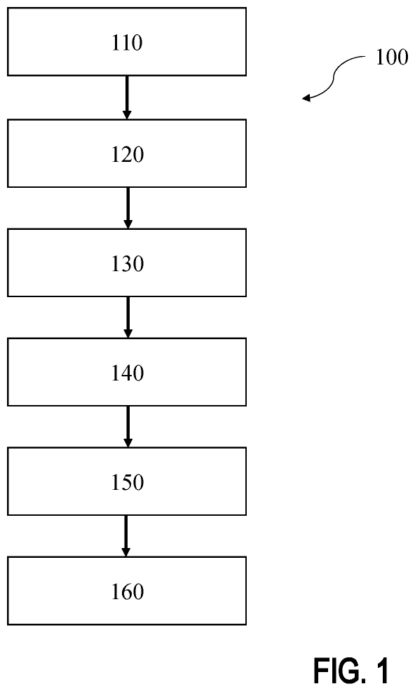

FIG. 1 shows an exemplary flow chart for monitoring 100 a health risk for a patient, in particular a PAD vs. a PVD venous disease. A commonly used monitoring flow is called “VESSEL” which is represented by the exemplary flow chart depicted in FIG. 1. Therein, each of the steps 110-160 corresponds to one letter of the term “VESSEL” and each of the steps of method 100 intends to distance a potential PAD from a PVD, i.e., during each step of 100 individual characterization steps can be carried out to distance a potential PAD from a PVD. Generally, arteries carry oxygen-rich blood away from the heart whereas veins return oxygen-poor blood back to the heart. The individual steps to be performed during each of the method steps 110-160 is outlined below.

The monitoring method can start with step 110 corresponding to the letter “V”. Step 110 intends to characterize various positions that can help alleviate discomfort/pain for a patient.

As an example, to investigate whether a patient suffers from PAD, it can be characterized whether dangling of the legs downward (dependent position) helps to decrease potentially prevailing pain. In this context, it can further be elicited whether an elevation of the legs causes a deterioration of the experienced pain.

In case of investigating whether a patient suffers from a venous disease, it can be characterized whether an elevation of at least one leg can decrease swelling of the at least one leg and whether the elevation of the at least one leg contributes to an improvement of a blood flow through the at least one leg. Further in this regard, it can be characterized whether a dangling of the at least one leg or standing/sitting for long periods of time (e.g., for several minutes, for several hours, etc.) leads to an increase of the pain and a potential edema.

Subsequently, in step 120, corresponding to the letter “E” an explanation for the pain can be investigated. This can include a characterization under which circumstances the pain can be experienced and how the pain can be experienced (e.g., sharp, etc.).

In case of investigating whether a patient suffers from PAD, it can be characterized whether the pain is experienced as sharp (e.g., worst at night), whether the patient experiences “rest pain” (e.g., the patient wakes up from sleep with pain (when the legs are in the horizontal position it can impede blood flow) and whether the patient dangles the extremity off the bed to alleviate the pain. Additionally or alternatively, it can be investigated whether an intermittent claudication is present. This can include a characterization whether an activity (e.g., running, walking etc.) causes severe pain in the calf muscles, thighs, buttocks etc. whereas, and whether, when the activity is stopped, the pain eases up. This can be due to fact that the muscle is being deprived of blood flow due to a prevailing PAD which can then cause the experienced pain.

In case of investigation whether a patient suffers from a venous disease (PVD), it can be characterized whether the pain is experienced as heavy, dull, throbbing or achy. Moreover, it can be characterized whether the pain is experienced worst when standing or sitting for long periods. Additionally or alternatively it can be characterized whether elevating legs eases the pain and potentially existing swelling.

Subsequently, in step 130, corresponding to the letter “S”, a skin of a lower extremity (e.g., at least one leg of the patient) can be characterized, preferably with respect to its color and/or temperature.

In case of investigating whether a patient suffers from PAD, it can be characterized whether the skin is thin, dry/scaly, hairless (or at least loss of hair at at least one extremity (such as, e.g., at least one leg)) and whether thick toenails can be observable. In some cases, it can further be characterized whether a dangling of at least one leg leads to a rubor of the at least one leg and whether an elevation of at least one leg leads to a paling (or bluish skin color) of the at least one leg. Additionally or alternatively, it can be possible that the patient suffers from sores or wounds on at least one toe, at least one feet or at least one leg that will not heal.

In case of investigating whether a patient suffers from a venous disease, it can be characterized whether at least one leg shows a thick, tough skin and/or whether the at least one leg shows a brownish color.

Subsequent, in step 140, corresponding to the letter “S”, a strength of pulse in at least one lower extremity can be characterized.

In case of investigating whether a patient suffers from PAD, it can be characterized whether the pulse is very low or even absent. This can occur to the fact that if a PAD is prevailing, the blood flow to at least one extremity can be decreased.

In case of investigating whether a patient suffers from a venous disease, it can be elicited whether a pulse is present (and preferably whether the pulse is normal). This can be explained due to the aspect that in case of a prevailing venous disease, there can generally not be a blockage in the blood flow from the heart to the at least on extremity. Instead, a blockage can be present in the return path of the blood flow, e.g., from the at least one extremity to the heart.

Subsequently, in step 150, corresponding to the letter “E”, the presence of an edema can be investigated.

More specifically, in case of investigating whether a patient suffers from PAD, it can be uncommon that an edema is present.

However, with a view to a potentially present venous disease, an edema can be present, and it oftentimes tends to deteriorate towards an end of a day.

Subsequently, in step 160, corresponding to the letter “L”, the presence (and preferably the appearance) of a lesion can be characterized.

In case of investigating whether a patient suffers from PAD, it can be characterized whether a lesion (e.g., an ulcer) is present. If this is the case, its location can be characterized. In case of a present PAD, the lesion can most likely be located at the end of at least one toe (e.g., close to a respective toenail), at the top of at least one foot (e.g., dorsum) and/or at a lateral ankle region (e.g., malleolus) of at least one foot.

It can further be characterized whether any kind of drainage is present (e.g., very little drainage), whether some (e.g., little) tissue granulation (e.g., pale/very light pink) is present or whether the tissue granulation is necrotic/black. Further in this context, it can be elicited whether a potentially present ulcer is deeply “punched out” with noticeable margins/edges that give it a round-shaped appearance.

In case, if it is investigated whether a patient suffers from a venous disease, it can also be characterized whether a lesion (e.g., an ulcer) is present and if this is the case, whether the ulcer is located at at least a medial part of a lower leg and/or a medial ankle region of a leg of the patient. Moreover, the appearance of the ulcer can be characterized. In this regard, it can be characterized whether the ulcer appears as swollen with a drainage, if a granulation is present (e.g., colored between deep pink to red) and whether the edges of the ulcer are irregular with a shallow depth.

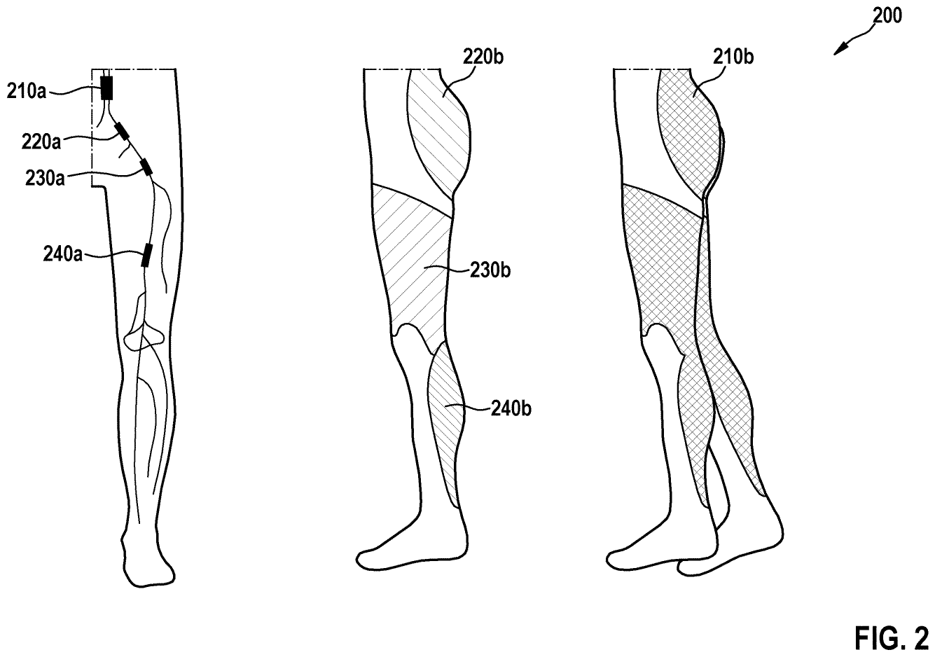

FIG. 2 exemplarily illustrates the conjunction 200 between a potential claudication (e.g., a stenosis or occlusion) of at least one artery in a leg of a patient and the associated location of pain experienced (corresponding to the letter “E” of the term “VESSEL” as outlined with reference to FIG. 1, above) by the patient as a result of a stenosis or occlusion of a particular artery.

A claudication can typically be experienced by a patient as an aching or burning in at least one leg muscle. Moreover, the pain can reliably be reproduced after walking for a certain distance and can be relieved within minutes of rest. Moreover, in case of a present claudication, pain can never be present at rest and the pain can not be exacerbated by certain positions/orientations of the leg.

If a claudication as such is present, the location of the pain can provide an indication of the site of the disease, e.g., which artery can experience a stenosis and/or occlusion.

As an example, a stenosis or an occlusion of an aorta 210a of a leg of a patient can cause a claudication at a bilateral buttock, a thigh and a calf 210b.

A stenosis or an occlusion of common iliac artery 220a can lead to pain experienced in a buttock area 220b of a leg of a patient.

A stenosis or an occlusion of a common femoral artery 230a can lead to pain experienced in a thigh area 230b of a leg of a patient.

A stenosis and or an occlusion of a superficial femoral artery 240a can cause pain in a calf area 240f of a leg of a patient.

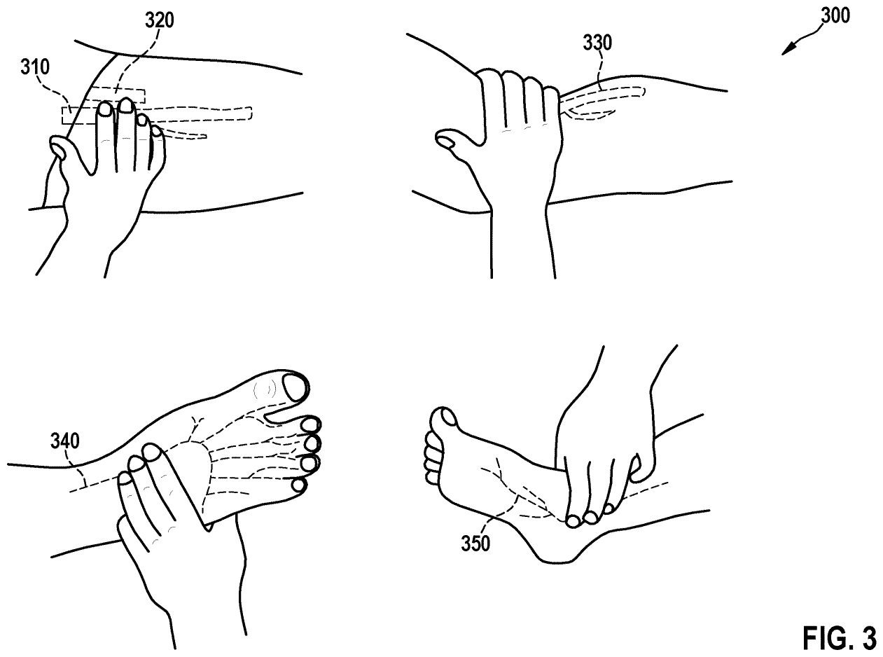

FIG. 3 exemplarily shows different locations 300 at which the strength of a pulse of a patient can be determined. It is noted that a strength of a pulse can thus be associated with a location at which the pulse was determined. This location-specific determination of the pulse of a patient can provide additional information on the potential prevalence of a PAD, venous disease and/or a diabetes.

It can thus be possible determine a strength of the pulse of a patient in the thigh of a leg of a patient such as in the femoral artery 310 of a patient. Additionally or alternatively, it can be possible to determine a strength of a pulse in a femoral vein 320 of a patient.

Additionally or alternatively it can be possible to determine a strength of a pulse of a patient in a popliteal cavity of a leg of a patient such as in a popliteal artery 330 of a patient.

Additionally or alternatively, it can be possible to determine a strength of a pulse of a patient at a feet of a patient, e.g., in a dorsalis pedis artery 340 of a patient.

Additionally or alternatively, it can also be possible to determine a strength of a pulse of a patient in the posterior tibial artery 350.

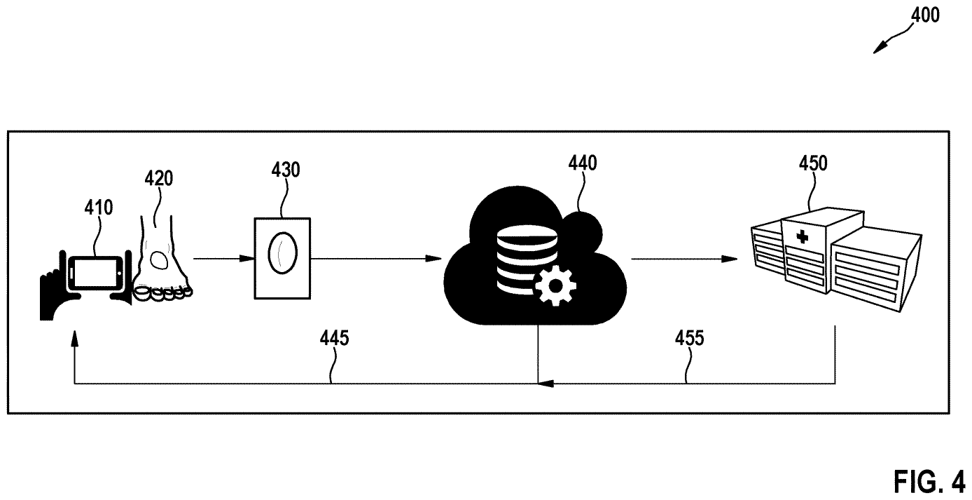

FIG. 4 exemplarily shows a system 400 overview of communicating devices according to an aspect of the present invention.

The system 400 can include a local device 410 which can be adapted with a module for obtaining visual information 420 of a skin of a patient.

The visual information 420 can include visual information associated with a foot and/or a leg of a patient and/or a hand and/or an arm of the patient.

In some cases, the visual information 420 can be or can be converted into a video sequence 430. The video sequence 430 can be forwarded to a remote entity 440. The forwarding of the video sequence 430 can preferably occur in an encrypted manner.

The remote entity 440 can include a cloud-based system. The remote entity 440 can include a module for storing at least the video sequence 430 received from the local device 410. The remote entity 440 can include an artificial intelligence (AI) engine for at least partially processing the received video sequence 430. The processing of the video sequence 430 can include detecting an anomaly in the video sequence 430 and/or assigning a scoring value to a potentially detected anomaly.

If an anomaly is detected and/or if a prevalence of a health risk for a patient is determined, an indication 445 can be provided to the local device 410. The indication can include, e.g., a scoring value assigned to a detected anomaly and/or answers to questions associated with the health state of the patient. Additionally or alternatively, the indication can warn a patient that a potential existing health risk has deteriorated, that a health risk was detected for the first time and/or that medical supported can be indicated.

The remote entity 440 can further include a module communicating with a clinical system 450.

The clinical system 450 can include a hospital information system, one or more clinicians (e.g., a doctor, a nurse, scientists, etc.). By the clinical system 450, it can be facilitated that a doctor and/or a nurse of the patient can monitor a potential health risk for a patient based at least in part on one or more video sequences 430 received from the remote entity 440.

Additionally or alternatively, the data received at the clinical system 450 (from the remote entity 440) can be provided to at least one scientist who can thus gain access to the at least one video sequence 430 for the sake of studying the properties/signatures of PAD, PVD and/or diabetes.

The data provided to the clinical system 450 can be limited to at least one video sequence 430. In some cases, the data provided to the clinical system 450 can further include a scoring value associated with a prevalence of a health risk for the patient.

In some cases, a doctor, a nurse and/or a scientist can interpret the data received from the remote entity 440 and can determine, e.g., that an error occur in the assigning of a scoring value and/or that an anomaly was detected erroneously. In such a case, feedback 455 can be provided to the remote entity 440 such that the AI engine of remote entity 440 can be updated based on the feedback 455. This can facilitate a more accurate determination of an anomaly and/or a more accurate assignment of a scoring value for subsequent video sequences 430.

Additionally or alternatively, the feedback 455 can include a direct feedback, sent by a doctor, a nurse and/or a scientist, to a patient. The feedback 455 can be received by the local device 410 and can be indicated to the patient. The feedback can include statement by, e.g., a doctor that no health risk is present, that a potentially pre-known health risk has deteriorated or can include a warning that a patient should seek for medical assistance. In some cases, the feedback 455 can include a request for an additional video sequence 430.

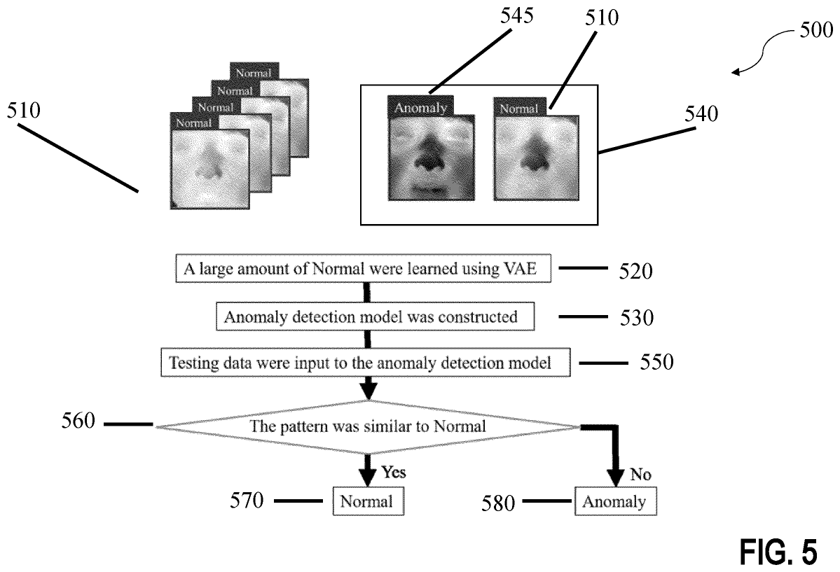

FIG. 5 shows an exemplary flow diagram of an anomaly detection method 500 according to an aspect of the present invention and using the AI engine as outlined above.

Prior to detecting an anomaly, normal data sets 510 (e.g. of healthy patients) are supplied to the aforementioned AI engine for training the AI engine (e.g., AI engine 440). Based thereon, the AI engine can learn to encode and/or reconstruct the normal data sets 510, wherein the training algorithm can, e.g., at least in part be based on a variational autoencoder (VAE), a generative adversarial network (GAN) and/or a convolutional neural network (CNN or ConvNet). Additionally or alternatively, a vector quantized variational autoencoder-2 (VQVAE-2) can be used.

After the AI engine has successfully learned 520 (has been trained, respectively) a large amount of normal data sets 510, the underlying AI model can be considered as a constructed anomaly detection model 530.

For testing the performance of the trained AI engine (e.g., its capability to predict an anomaly when an anomaly is present in a data set supplied to the trained AI engine and its capability not to predict an anomaly when no anomaly is present in a data set supplied to the trained AI engine), a test data set 540 can be provided to the AI engine.

The test data set 540 can include at least one representative of the training data set 510 (and/or an additional data set considered as normal which was not part of the initial training data set) and one or more data sets 545 which can include an anomaly.

The test data set 540 can then be provided 550 to the trained AI engine.

Based thereon, the trained AI engine can decide 560 whether the training data set 540 is similar to the normal data sets 510 used for training the AI engine. If the training data set is similar 520 to the normal data set 510, the training data set can also be considered as normal, i.e., as not including an anomaly.

However, if the trained AI engine decides 560 that the training data set 540 includes is not similar to the normal data set 510, an anomaly 580 can be determined.

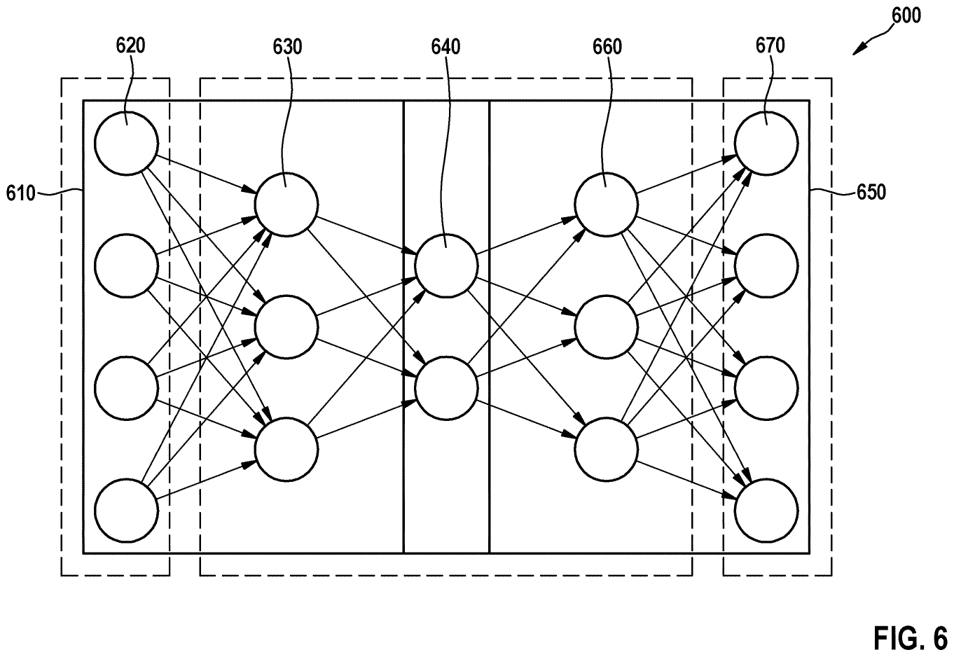

FIG. 6, shows an exemplary neural network 600 as it can be used as an autoencoder (AE).

The AE can include an encoder 610, exemplarily including an input layer 620, a first hidden layer 630 and a second hidden layer 640. Since the number of nodes in subsequent layers decreases from the input layer 620 to the second hidden layer 640, this transition is accompanied by a loss of information as will further be described below.

The encoder 610 can be followed by a decoder 650, exemplarily including, the second hidden layer 640, a third hidden layer 660 and an output layer 670. Since the number of nodes per layer increases from the second hidden layer 640 to the output layer 670, the amount of information which can be stored in each of the layers increases as it will further be described below.

The input layer 620 can exemplarily include four nodes. Input layer 620 can be configured to represent an input image to be processed by the exemplary neural network 600. It is noted that the four nodes of input layer 620 are only shown for the sake of simplicity and that in real applications, the number of nodes in the input layer 620 can typically higher. As an example, an image with a resolution of 1024×768 Pixel can be transformed into a single 1 by x vector including 1024×768=786,432 rows each row exemplarily representing a greyscale value between 0 and 255. It is emphasized that the neural network 600 is not limited to the processing of greyscale images only and can also be configured to process color images, e.g., in the RGB space, the YMCK space and/or any other suitable color space. If an image represented in the RGB space is provided to the input layer 620, the image can exemplarily be transformed into a vector with 1024×768×3=2,359,296 rows. Here, the factor of three accounts for the representation of the respective image in the RGB colors space including three basis colors. The exact value of each entry in the vector accounts for the brightness of a pixel in, e.g., the red colors space. In other words, a first set of 786,432 (consecutive) rows can represent the red colors space (and each row can represent the brightness of a particular pixel in the red color space), a second set of 786,432 (consecutive) rows can represent the green color space (and each row can represent the brightness of a particular pixel in the green color space) and a third set of 786,432 (consecutive) rows can represent the blue colors space (and each row can represent the brightness of a particular pixel in the blue color space). It is further noted that the architecture of the input layer 620 is not limited to these examples but can also be configured with any other suitable architecture.

Each of the nodes of the input layer 620 can be interconnected to each of three exemplary nodes assigned to the first hidden layer 630. Each interconnection can be accompanied by a weighting factor multiplied to the data propagated between two (subsequent) nodes (i.e. from a node of the input layer 620 and a node of the first hidden layer 630). Since the first hidden layer 630 can only include three nodes as compared to the four nodes assigned to the input layer 620, the amount of information which can be represented by the first hidden layer 630 can be lower as compared to the amount of information which can be represented by the input layer 620. Therefore, the transition from the input layer 620 to the first hidden layer 630 can be understood as a dimension reduction of the image input into the neural network 600. The associated loss of information can, e.g., lead to one or more of a loss of color (e.g., a transition from a colored image to a black and white image, a loss of contrast, a loss of certain boundaries/margins in the image, etc.).

Each of the three nodes of the first hidden layer 630 can be interconnected with each of two nodes (only shown exemplarily) assigned to the second hidden layer 640. Each interconnection can be accompanied by a weighting factor multiplied to the data propagated between two (subsequent) nodes. Since the amount of information which can represented by the second hidden layer 640 as compared to the first hidden layer 630 is reduced (due to a reduced number of nodes), the transition from the first hidden layer 630 to the second hidden layer 640 can again be understood as a dimension reduction. This can again be understood as a loss of information of the image processed by the neural network 600. The output of the second hidden layer 640 can be referred to as a reduced image.

After removing features from the image fed into the input layer 620 (which can be associated with the encoder 610), a processing of the originally presented image by the decoder 650 can be performed.

Since the number of nodes increases with each layer included by the decoder 650, the amount of information storable/processible in each of the layers can increase. This concept can be used by the neural network 600, and in particular by the layers/nodes assigned to the decoder 650, to reconstruct the originally presented image (as fed into the input layer 610) from the reduced image generated at the second hidden layer 640.

This can, inter alia, be done adding additional features to the reduced image in each of the third hidden layer 660 and the output layer 670, e.g., by adding color information to the image, contrast information, boundaries/margins, etc.). This can be encoded in respective weighting factors associated with an interconnection of two nodes in respective subsequent layers.

The image obtained at the output layer 670 can then be compared to the image originally provided to the input layer 620. The comparing can include the calculation of an error function wherein the absolute value of the error function can be large if the deviation between the image provided at the output layer 670 and the image originally fed into the input layer 620 is large (i.e., the image provided at the output layer 670 is significantly different from the image originally provided to the input layer 620, wherein certain features of the original image are not present in the reconstructed image and/or wherein features appear in the reconstructed image which were not present in the original image).

The applied training algorithm can then try to determine the associated weighting factors such that the error function can be minimized, i.e., the weighting factors are considered to be at their respective optimum if the image provided at the output layer 670 is nearly identical (including at most negligible differences, e.g., within the average noise level) to the image originally provided to the input layer 620. The training can be considered successful and/or complete, if the value of the error function is below a predefined threshold, preferably close to 0.

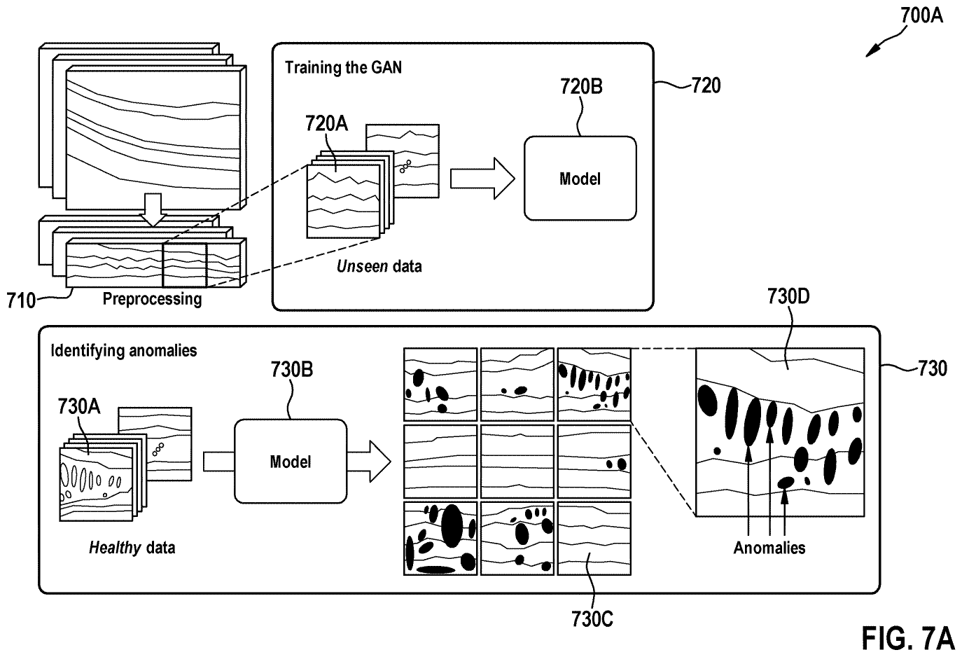

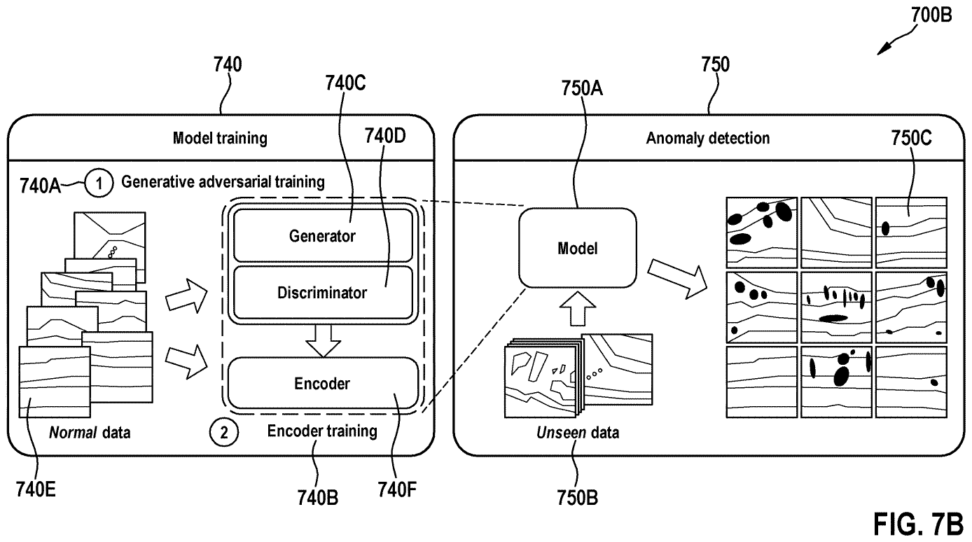

FIGS. 7A and 7B exemplarily illustrate the training process of a GAN used for determining an anomaly in obtained visual information according to an aspect of the present invention.

Notably, FIG. 7A illustrates a high-level overview of an exemplary training of a GAN and an exemplary determination of a potential anomaly in the obtained visual information.

Raw data (i.e., obtained visual information such as, e.g., the visual information 420 as described with reference to FIG. 4, above) can be preprocessed (e.g., color adjustments, contrast adjustments, sharpening filters, cropping, etc.) to obtain a training data set 710.

The training data set 710 can be provided to the GAN training 720. The GAN training 720 can be based on a set of preprocessed images 720A which can be considered as healthy, i.e., which do not include an anomaly, and a model 720B (e.g., included by the AI engine as described with reference to FIGS. 4 and/or 6, above) to be trained.

The GAN training 720 can exemplarily be based on the training of a generator. A generator can be based on a neural network, e.g., the generator can be based on layers 640-670 of exemplary neural network 600 as described with reference to FIG. 6, above (but it can also be implemented differently, e.g. including another generative neural network). Based thereon, the neural network of GAN training 720 can be trained to reconstruct (or more generally: generate) an artificial image which can be identified (e.g., by a discriminator as further outlined below) as a representative of the preprocessed images 720A.

If the training has successfully been completed, an anomaly detection 730 can subsequently be initiated based at least in part on the trained model 720B. The anomaly detection 730 can include providing a set of yet unseen data 730A (i.e., data which has not been used for training the model 720B) to the trained model 730B, identifying potential candidates 730C for an anomaly and determining 730D that the potential candidates 730C represent an anomaly.

Trained model 730B can additionally (as compared to the trained model 720B) include a trained encoder, which e.g., can be based on a neural network architecture similar to the architecture of layers 620-640 of the neural network 600 as described with reference to FIG. 6, above).

If unseen data is provided to the trained model 730B, the unseen data can first undergo an encoding (as described with reference to FIG. 6, above, and a subsequent reconstruction by a module of the trained generator included by the trained model 720B. However, potential anomalies present in the unseen data 730A can not be properly reconstructed by the trained model 730B as the underlying neural network has not been trained for reconstructing anomalies present in provided data sets. More specifically, the neural network has not been trained to reconstructed anomalies in the unseen data 730A.

Identifying potential candidates 730C for an anomaly can include comparing the reconstructed image of the unseen data 730A to an image of the training data 710 and/or to the unseen data 730A originally provided to the trained model 730B. Any deviation identified from the training data thereby can be understood as indicators for the presence of a potential anomaly in the unseen data 730A. Only deviations can be considered which exceed a predefined noise level. Noise in this case can be understood as disturbances in provided data (e.g., in the training data 720A and/or the unseen data 730A) which are not related to the remaining content of the respective data. A potential anomaly can only be seen as an anomaly if it can be separated from noise (e.g., a potential anomaly can only be seen as a potential anomaly if it can be represented by more than 10 adjacent pixels and/or by a certain area in an image (including the potential anomaly) which appears darker than surrounding areas in the image). The thus effectively arising difference an anomaly has to have as compared to image data not representing an anomaly can depend on the progression of the potential disease. In other words, a potential disease, monitored in an early stage, can be represented by a potential anomaly which only lies slightly above the noise level whereas a progressed disease can be represented by a potential anomaly which more clearly differs from the noise level as an anomaly associated with an early stage of a disease.

The deviation can, e.g., be expressed by the value of an error function. If the value of the error function exceeds a predefined threshold, a potential anomaly can be referred to as a determined anomaly 730D.

FIG. 7B shows the GAN training process as described with reference to FIG. 7A in more detail.

GAN training 740 can include generative adversarial training sub-step 740A and can include encoder training 740B.

Sub-step 740A can include a generator 740C and a discriminator 740D (considered as the adversarial). Generator 740C can be implemented as the generator described with reference to FIG. 7A, above.

In the following, the training of generator 740C in the context of a generative adversarial training can be described. Generator 740C can generate an artificial image (based on input data (e.g., random values, noise, encoded visual information and/or any other suitable input parameter) provided to the respective one or more input layers of generator 740C) which can represent visual information according to an aspect of the present invention. Said generated image is passed to the discriminator 740D which can determine if there is a difference between the artificially generated image and a real image 740E (considered as healthy, i.e., not including an anomaly, which can be included by the training data set 720A), e.g., based on calculating a value of an error function (and/or a rewards function) as outlined above. The value of the error function can be fed back into the generator 740C and the generator 740C can generate, based at least in part on the value of error function, a new artificial image which can then again be passed to the discriminator 740D. This process can iteratively be repeated until the value of the error function is below a predefined threshold. In this case, the generative adversarial training sub-step 740A is considered to have successfully been terminated.

Subsequent to the generative adversarial training sub-step 740A, encoder training 740B can be carried out. Encoder training 740F can take advantage of the generative adversarial training sub-step 740A and an encoder 740F.

Encoder training 740B can include training an underlying neural network such that a dimension reduction of the visual information provided to the encoder can occur and such that the input data provided to generator 740C can be obtained as an output of the encoder 740F. The encoder 740F can be implemented as the encoder described with reference to FIG. 6, above. The training of the encoder 740F can be based at least in part on the training of a neural network as described elsewhere herein (e.g., based on a minimization of an error function).

If the encoder 740F has successfully been trained, the actual anomaly detection 750 can follow. Anomaly detection 750 can include providing unseen data 750B (i.e., data which has not been part of the training data 740A), which can include an anomaly to be detected, to the trained model 750A. The trained model 750A can be based on the trained generator 740C and the trained encoder 740F.

To determine whether an anomaly is present in the unseen data 750B, unseen data 750B (or at least one representative of unseen data 750B, such as, e.g., an unseen image) can be provided to the trained model 750A. The provided unseen data 750B can undergo a dimension reduction by the trained encoder 740F. After the encoding, the encoded unseen data 750B can be provided to the trained generator 740C as input data based on which the trained generator 740C can generate an artificial image. The generated artificial image can be compared to the unseen data 750B originally provided to the encoder 740F. Any difference (wherein a difference can be defined as described elsewhere herein) derivable from said comparison can be understood as an indication for a potential anomaly in the unseen data 750B as both the encoder 740F and the generator 740C have only been trained to encode and reconstruct (generate) real images 740E considered as healthy (i.e., not including an anomaly). Additionally or alternatively, it can also be possible that the generated artificial image is compared to one or more images of real images 740E to derive an indication for a potential anomaly in the unseen data 750B. A potential anomaly in the unseen data 750B can be seen as an anomaly if the aforementioned difference exceeds a predefined value (which preferably exceeds the noise level in the generated artificial image).

It is further emphasized that a key aspect of training a GAN can be seen in obtaining a trained generator 740C which is optimized (due to the training) to provide an image which will be accepted by a discriminator 740D. In other words, a trained generator 740C of a GAN can be optimized to generate an artificial image which can be that close to a real image 740 that it can not be rejected by discriminator 740D (e.g., such that it can not be distinguishable from a real image 740 for the discriminator 740D).

In comparison to that, a trained auto-encoder can be optimized to memorize how to reconstruct an originally presented input data set in greatest detail and efficiency.

The GAN-based approach for anomaly detection and the autoencoder-based concept for anomaly detection can be implemented as alternative solutions. Alternatively, it can also be possible to combine the GAN-based approach with the autoencoder-based approach (e.g., the layers 640-670 of the neural network 600 as described with reference to FIG. 6 can be replaced by generator 640C and can be trained according to an interplay of a respective generator and a respective discriminator).

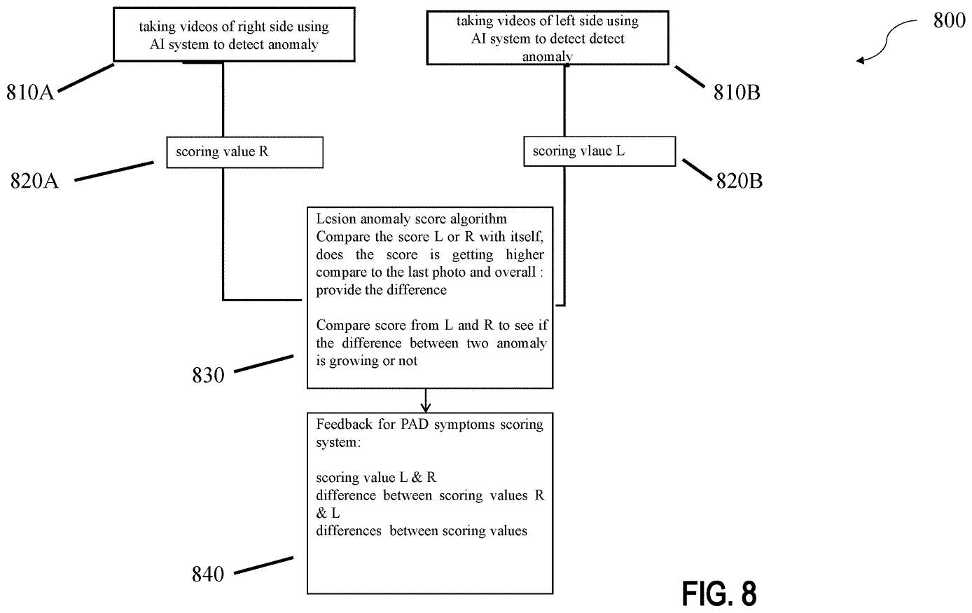

FIG. 8 shows an exemplary flowchart of a method 800 for detecting that at least one side of a body of a patient is developing a PAD (and/or a PVD and/or a diabetes).

The method 800 can be based on a first step 810A obtaining visual information associated with a skin of a patient and determining whether the visual information includes an anomaly according to aspects as presented herein. The step 810A can be carried out for a first side (e.g., a right side) of the body of the patient (such as, e.g., a right leg of the patient).

The method 800 can further include obtaining visual information associated with a skin of the patient and determining whether the visual information includes an anomaly according to aspects as presented herein. The step 810B can be carried out for a second side (e.g., a left side) of the body of the patient (such as, e.g., a left leg of the patient).

Based on the determination that the obtained visual information includes an anomaly, a scoring value can be assigned to a respective right side of the body of the patient (scoring value 820A) and to a respective left side of the body of the patient (scoring value 820B).

The individually assigned scoring values (820A, 820B) can then be forwarded to a scoring comparison algorithm 830. The scoring comparison algorithm 830 can compare each of the scoring values 820A and 820B to respective previously determined scoring values (e.g., historic data) associated with previously obtained visual information. This can allow to determine a temporal evolution of the previously assigned scoring values to the presently assigned scoring values and can thus allow to determine whether a deterioration of an anomaly has occurred over time. An increase between a currently assigned scoring value 820A and 820B, respectively, can be an indication for a deterioration of a health risk for a patient, e.g., of a PAD and/or a PVD and/or a diabetes.

Moreover, scoring comparison algorithm 830 can further be configured to compare the currently assigned scoring values 820A, 820B to each other to determine a numerical difference between the currently assigned scoring values 820A, 820B. The determined numerical difference can be compared to previously determined numerical differences to determine how the difference between assigned scoring values 820A and 820B can have changed over time.

The method can only include step 810A or 810B, and the scoring comparison algorithm 830 can only compare the scoring value 820A or 820B to respective previously determined scoring values (e.g., historic data) associated with previously obtained visual information. This can allow to determine a temporal evolution of the previously assigned scoring value(s) to the presently assigned scoring value and can thus allow to determine whether a deterioration of an anomaly has occurred over time.

The calculation results of scoring comparing algorithm 830 can be provided as feedback 840. Feedback 840 can include the currently assigned scoring values 820A and/or 820B. The feedback can additionally or alternatively include the difference value between the currently assigned scoring values 820A and/or 820B. Additionally or alternatively, the feedback 840 can include one or more difference values between respective currently assigned scoring values 820A and/or 820B and previously assigned scoring values.

In addition to the scoring value 820A and/or the scoring value 820B, a comparison scoring value can be determined to describe the change over time of the scoring value 820A and/or the scoring value 820B and/or the change of the scoring value 820A, respectively, the scoring value 820B in comparison to each other.

As outlined above, the determining whether the obtained visual information includes an anomaly can be followed by providing the patient with at least one question concerning the health state of the patient. The potentially received replies to the at least one question can then be assigned a weighting factor to estimate the association of the received replies with a certain disease (e.g., a PAD, PVD or a diabetes).

The most common symptom of PAD is intermittent claudication (IC), which is muscle pain or discomfort in the legs and/or buttocks brought on by walking and relieved within minutes on rest. IC occurs due to an inability to sufficiently increase blood flow (and oxygen de-livery) to match the metabolic demands of the lower-limb muscles during exercise the lower-limb.

The walking distance or speed at which symptoms occur can depend on multiple factors including the severity and site of the arterial disease. Therefore, higher scores can be assigned in the “Explanation of the pain section” of questionaries associated with the “VESSEL” approach as described above.

Providing the patient with at least one question concerning the health state of the patient can be done by a weighting algorithm which can, e.g., be implemented in an app, e.g., operating at the local device and/or at the remote device as described above. Table 3 as recited below states several exemplary questions/topics addressed by the at least one question and respective weighting coefficients (to be assigned based at least in part on the contentual information included in a response to a provided question) to support the monitoring of a health risk for a patient (preferably a prevalence of PAD).

TABLE 3
Exemplary questions/topics provide to the patient and respective
weighting coefficients to monitor a health risk for a patient.

| exemplary question/topic | weighting coefficient |
|---|---|
| Various positions that help alleviate | Every YES gets 1, every NO gets Zero |
| discomfort/pain | |
| Explanation of the pain (when it | Every YES gets 2 except: YES to |
| happens, characteristics of the pain) | question” Do you have pain at rest” gets |
| | score 4 |
| | Pain walking less than 200 m gets 3 |
| | Pain walking more than 200 m gets 2 |
| Skin (color, temperature, nails) | Every YES gets 1 score |
| | Answer “cold“ to temperature gets 2 |
| | Answer: ruber or red gets 2 score |
| | Answer: pale gets 2 score |
| Strength of Pulse | Answer NO gets 2 points |
| Edema | Answer NO, gets 0 points |
| Lesions (ulcers and their location and | Feedback for PAD symptoms scoring |
| appearance). | system |

When the patient starts using the app for the first time, he/she can reply to one or more of the aforementioned questions. The answers will be the base for comparison for the future when the patient can use the app on the specified intervals (e.g., periodically).

The weighting algorithm can be configured to first characterize the “E” of “VESSEL” and can thus provide at least one question to a patient which can be associated with an “Explanation of the pain” and can characterize a pain as the first clinical symptoms of PAD.

The limit for the system can be reached when the weighting coefficient in “Explanation of the pain” is 6. In this case, the patient can automatically be flagged for further tests without further checking of other symptoms. If the score for this section can be below 6 (and optionally at least 2 or at least 3), then the system can look at weighting coefficients associated with Lesions, as outlined herein. Next, it can move to other sections and their scores and can compare them with previously assigned weighting factors.

If at least one weighting coefficient for other sections gets higher over time, then the patient can be seen as more likely developing a PAD. If for example after 6 months of monitoring a health risk for a patient, at least one assigned weighting coefficient, associated with symptoms, increases in at least one section other than “Explanation of the pain”, then the patient can be flagged for further tests by a doctor. The overall history of symptoms progress along with type of pain, location of pain could be provided to the patient or their doctors.

The aforementioned weighting algorithm can best be suitable to be used by a patient who has a history of a heart and/or a diabetes disease and can not be implemented as a consumer app (e.g., the app can be used for (professional) monitoring purposes only and/or can be exclusively used by clinicians such as, e.g., doctors). The symptoms weighting reports can be shared with doctor for review and decision making if a patient needs to be called in.

Based on how the weighting algorithms assigns the weighting factors to the symptoms, the weighting coefficient could be used for detecting a venous disease or a diabetes as both these diseases can have skin manifestation as well.

The following Tables 4 and 5 show two further exemplary question sets to be provided to a patient and associated weighting coefficients to be assigned to the received replies for a monitoring of a health risk for the patient according to the “VESSEL” scheme as outlined above.

More specifically, Table 4 states several exemplary questions to be provided to a patient for monitoring whether a patient develops a PAD and/or whether an already existing PAD has deteriorated. In analogy to Table 4, Table 5 states several exemplary questions to be provided to a patient for monitoring whether a patient develops a PVD and/or whether an already existing PVD has deteriorated. Moreover, the Tables 4 and 5 further indicates whether a question is directly related to PAD/PVD and potential weighting factors assigned to a response to a question provided to a patient.

TABLE 4
exemplary questions provided to a patient for
monitoring a health risk for the patient

| | | PAD disease- | PAD |
|---|---|---|---|
| | | related | disease |
| Vessel App | Symptoms | answers | weighting |
| Various positions | Does Dangling the legs down | YES | 1 |
| that help alleviate | (dependent position) help with the | | |
| discomfort/pain | pain? Y/N | | |
| | Does elevation of legs help with the | No | 1 |
| | pain? Y/N | | |
| Explanation of | Do you have pain when you wake | Yes | 1 |
| the pain (when it | up? (Yes/NO) | | |
| happens, | Do you have pain at night? Y/N | Yes | 1 |
| characteristics of | Do you have pain with activities | Yes | 1 |
| the pain) | (running, walking etc.) Y/N | | |
| | Do you have pain when you rest? | No | 0 |
| | (Yes/NO) | | |
| | How far can you walk before you | Less than | 3 |
| | have pain less or more than 200 m: | 200 m | |
| | | More than | 2 |
| | | 200 m | |
| | Is pain sharp or Heavy or dull | Sharp | 1 |
| | or throbbing or achy? | | |
| | Where is the pain? Calf or tight | Indicate the | |
| | or buttocks | location of | |
| | | pain | |
| Skin (color, | Is your skin on your hands/feet feel | warm | 2 |
| temperature, | cold or warm when you touch? | | |
| nails) | Is your hair growth on legs and feet | Yes | 1 |
| | or hands Reduced | | |
| | Are your toenails or nails Brittle and | Yes | 1 |
| | are slow to grow? | | |
| | Do you have Itchy skin or | Yes | 1 |
| | Shiny skin? | | |
| | Change in the color of the skin? | Yes | 1 |
| | turning blue or pale, brown or | Pale or Blue | 2 |
| | nothing? | | |
| | Do you have any sore or wound? | Yes | 1 |
| | What is the color of feet when you | Ruber or red | 2 |
| | dangle it? “No change, Ruber or red | | |
| | What is the color when you elevate | Gets Pale | 2 |
| | it: No change, get pale | | |
| Strength of Pulse | Do you have normal pulse or weak | Weak or no | 1 |
| | or no pulse | pulse | |
| Edema | Do you have Edema | No | 0 |
| Lesions (ulcers | Numerical feedback from PAD | | Add |
| and their location | system | | numerical |
| and appearance). | | | score |

The following table shows a further exemplary question set provided to a patient for monitoring a health risk for the patient to monitor a potential venous disease (e.g, PVD).

TABLE 5
exemplary questions provided to a patient for
monitoring a health risk for the patient

| | | Venous | |
|---|---|---|---|
| | | disease- | Venous |
| | | related | disease |
| Vessel App | Symptoms | answers | weighting |
| Various positions | Does Dangling the legs down | No | 1 |
| that help alleviate | (dependent position) help with the | | |
| discomfort/pain | pain? Y/N | | |
| | Does elevation of legs help with the | YES | 1 |
| | pain? Y/N | | |
| Explanation of the | Do you have pain when you wake | No | 0 |
| pain (when it | up? (Yes/NO) | | |
| happens, | Do you have pain at night? Y/N | No | 0 |
| characteristics of | Do you have pain with activities | No | 0 |
| the pain) | (running, walking etc.) Y/N | | |
| | Do you have pain when you rest or | Yes | 1 |
| | standing? (Yes/NO) | | |
| | How frequent is the pain: | Occasional | 2 |
| | Daily or Occasionally | Daily | 3 |
| | Is pain sharp or Heavy or dull | Dull or | 1 |
| | or throbbing or achy | throbbing | |
| Skin (color, | Change in the color of the | Brown | 1 |
| temperature, nails) | skin? turning blue or pale, | | |
| | brown or nothing? | | |
| | Do you have any sore or | Yes | 1 |
| | wound or ulcer | | |
| Strength of Pulse | Do you have normal pulse or weak | Normal | 1 |
| | pulse | | |
| Edema | Do you have Edema | Yes | 1 |
| | When? | | |
| | Evening only | Evening only | 1 |
| | Afternoon | Afternoon | 2 |
| | Morning | Morning | 3 |
| Lesions (ulcers and | Numerical feedback from PAD | | Add |
| their location and | system | | numerical |
| appearance). | | | score |

The questions as outlined above can be provided to both (potential) PAD and (potential) PVD patients.

By assessing the replies to the questions and the assigned scoring values (visual information), the weighting algorithm can be capable of assigning a diseased patient to a PAD and/or a PVD. It is noted that some symptoms are in common whereas other symptoms are completely different than others.

The difference between PAD and PVD weighting assessment can be that the weighting algorithm can start with questions associated with a pain and can then move to Edema. Afterwards it can focus on other symptoms in comparison with PAD.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A device for monitoring a health risk for a patient, comprising:
means for obtaining visual information associated with a skin of the patient;
means for detecting an anomaly on the skin of the patient based at least in part on the obtained visual information;
means for assigning a to the detected anomaly, wherein the scoring value is associated with the health risk for the patient.

2. The device of claim 1, wherein the means for obtaining comprise means for receiving the visual information from a local device.

3. A device for monitoring a health risk for a patient, comprising:
means for obtaining visual information associated with a skin of the patient;
means for receiving a scoring value to be assigned to an anomaly on the skin of the patient based at least in part on the obtained visual information, wherein the scoring value is associated with the health risk for the patient.

4. The device of any of claim 1, wherein the health risk is a prevalence, presence and/or progression of a peripheral arterial disease, PAD, and/or a peripheral vascular disease, PVD, and/or a diabetes.

5. The device of any of claim 1, wherein the visual information is a video sequence.

6. The device of claim 1, wherein the means for detecting detects a lesion at least one location on the skin of the patient and an appearance of the lesion.

7. The device of claim 1, wherein
the visual information comprises a first visual information and a second visual information, wherein the first visual information and the second visual information are associated with a part of the skin of the patient and the second visual information is obtained later in time than the first visual information;
the means for detecting an anomaly are configured to detect a first anomaly on the part of the skin at least in part on the obtained first visual information, and a second anomaly on the part of the skin at least in part on the obtained second visual information; and
the means for assigning a scoring value are configured to assign a first scoring value to the first anomaly and a second scoring value to the second anomaly; and the device further comprising:
means for making a comparison between the first scoring value and the second scoring value; and
means for assigning a comparison scoring value to the second anomaly based on the comparison.

8. The device of claim 1, wherein
the means for obtaining visual information are configured to obtain a first visual information associated with a first part of the skin and a second visual information associated with a second part of the skin;
the means for detecting an anomaly are configured to detect a first anomaly on the first part of the skin at least in part on the obtained first visual information, and a second anomaly on the second part of the skin at least in part on the obtained second visual information; and
the means for assigning a scoring value are configured to assign a first scoring value to the first anomaly and a second scoring value to the second anomaly; and
the device further comprising:
means for making a first comparison between the first scoring value and the second scoring value; and
means for assigning a first comparison scoring value to the first anomaly based on the first comparison.

9. The device of claim 8, wherein
the means for obtaining visual information are further configured to obtain a third visual information associated with the first part of the skin and a fourth visual information associated with the second part of the skin, wherein the third visual information and the fourth visual information are obtained later in time than the first visual information, respectively, the second visual information;
the means for detecting an anomaly are further configured to detect a third anomaly on the first part of the skin at least in part on the obtained third visual information, and a fourth anomaly on the second part of the skin at least in part on the obtained fourth visual information;
the means for assigning a scoring value are configured to assign a third scoring value to the third anomaly and a fourth scoring value to the fourth anomaly;
the means for making the first comparison are further configured to make a second comparison between the first scoring value and the third scoring value, and a third comparison between the second scoring value and the fourth scoring value, and/or a fourth comparison between the third scoring value and the fourth scoring value; and
the means for assigning the first comparison scoring value are further configured to assign a second comparison scoring value to the third anomaly based on the second comparison and the third comparison and/or the first comparison and/or the fourth comparison.

10. The device of claim 1, wherein the means for detecting an anomaly on the skin of the patient comprise an artificial intelligence engine.

11. The device of claim 1, comprising:
means for providing the patient with at least one question associated with the health state of the patient, wherein the at least one question is associated with an extent of a pain experienced by the patient and/or a location of the pain experienced by the patient;
means for receiving at least one answer from the patient in response to the at least one question.

12. The device of claim 11, comprising:
means for weighting the received at least one answer and/or the scoring value, respectively, the first scoring value and/or the second scoring value and/or the third scoring value and/or the fourth scoring value and/or the first comparison scoring value and/or the second comparison scoring value to provide an estimation of the prevalence of PAD, PVD and/or diabetes.

13. A method for monitoring a health risk for a patient, comprising:
obtaining a first visual information associated with a first part of a skin of the patient;
detecting a first anomaly on the skin of the patient based at least in part on the obtained first visual information;
assigning a first scoring value to the detected first anomaly, wherein the first scoring value is associated with the health risk for the patient.

14. The method according to claim 13, wherein the detection of an anomaly comprises:
providing the obtained first visual information to a trained artificial intelligence, engine, wherein the trained AI engine has been trained on previously obtained visual data considered as healthy and based on a skin type of the patient; and/or
deriving a first anomaly in the obtained first visual information through the trained AI engine by reconstructing the obtained first visual information by an autoencoder of the trained AI engine and comparing the reconstructed information to the obtained first visual information.

15. (canceled)

16. A computer program comprising code, which when executed on a computer, performs the method of claim 13.
